(19) United States
(12) Patent Application Publication
(10) Pub. No.: US 2004/0097466 A1
(43) Pub. Date: May 20, 2004

(54) Treatment of a patient suffering from cell surface thrombosis disorders using enhanced absorption of arginine

(76) Inventor: Bruce Daniels, Oklahoma City, OK (US)

Correspondence Address: Ali Kamarei, Esq., 280 Colorado Avenue, Palo Alto, CA, 94301, US

(21) Appl. No.: 10/299,506

(22) Filed: Nov. 19, 2002

(51) Int. Cl. A61K 31/727

(52) U.S. Cl. 514/56

(57) ABSTRACT

The invention as described comprises a method and composition for enhanced absorption of arginine in mammals, for the treatment of the cellular environment in cardiovascular diseases comprising the step of administering to a patient a therapeutically-effective amount of polyarginine and co-administering a therapeutically-effective amount of heparin or its functional analog or their physiologically acceptable salt. The dose of polyarginine administered to the patient is between approximately 100 mg to 6000 mg daily. Optionally, the dose of polyarginine administered to the patient is between approximately 200 mg to 1900 mg daily. Optionally, the dose of polyarginine administered to the patient is between approximately 400 mg to 1800 mg daily.

### FIELD

This invention relates to a pharmacological composition and method that is directed to a patient susceptible to or suffering from a cardiovascular disorder or disease, and more particularly, but not by way of limitation, to a formulation with enhanced absorption characteristics for preventing and treating atherosclerosis, arteriosclerosis, congestive heart failure, arterial stenosis, cardiac cell hypertrophy, thrombogenicity, myocardial infarction, cerebrovascular ischemia, peripheral vascular ischemia, angina pectoris, hypertension or endothelial dysfunction.

### BACKGROUND

Cardiovascular disorders and diseases resulting from cell surface thrombosis, and their associated complications are a principal cause of disabilities and deaths of individuals in the world. For example, in recent years more than 500,000 deaths have occurred annually in the United States alone as a result of coronary artery disease, and an additional 1,200,000 patients have been hospitalized for myocardial ischemia and infarction.

There has been significant and extensive research for effective long term treatment for disorders and diseases of the heart and arteries, such as atheresclerosis, arteriosclerosis, congestive heart failure, angina pectoris, and other diseases associated with the cardiovascular system. However, present treatments for such disorders are short term treatments such as administration of vasodilators, angioplasty, and by-pass surgery. These treatments have serious shortcomings in long-term effectiveness, thus they have met with great disapproval due to the risks associated with them. The use of vasodilator drugs and mechanical treatments for acute and chronic occlusive vascular diseases of the heart central and peripheral vascular systems have to date been ineffective for favorable long-term results and do not treat the underlying molecular processes recognizable for the disease.

The focus of current treatment methods is to react to potentially immediate danger to one's life. Even the prescription of “statin” drugs such as Lovastatin, were originally designed to treat patients with significant risk of present danger of heart attacks due to high cholesterol levels. The only reason the long term risks associated with taking cholesterol reducing agents or “statins” was justified because of the immediate danger the high cholesterol levels presented to a patient. Almost all of the current treatment methods focus on reducing and/or eliminating the occlusion of larger arteries and none take into consideration that for example over 75% of fatal heart attacks are in patients with no present signs of significantly occluded arteries. The insertion of stents and such mechanical devices into larger arteries to prevent occlusion are only temporary procedures. Thus, the result is that myocardial infarction is temporarily delayed. However, such procedures merely postpone eventual myocardial infarction as the underlying processes continue untreated. The result of the current treatments has had minimal impact on the long-term processes of atherosclerosis. For example a significant number of patients who receive angioplasty have a repeat coronary event within three to five years. The cost associated with these treatments, both in terms of medical expenses as well as fatalities and lost productivity, is enormous.

Furthermore, the rationale for using statin drugs to lower plasma cholesterol fails to explain why coronary heart attacks generally occur in individuals with non-critical blockages and why blockages do not occur in capillaries or veins. Even when used, statin drugs reduce the risk of a recurrent coronary event, only by 30 to 40%.

The rationale for vasoactive drugs is to reduce blood pressure by acting directly or indirectly on vascular and/or cardiac smooth muscle, thereby decreasing vascular resistance to flow. Such drugs do not treat initial cause of elevated pressure and abnormal flow. Rather, they seek to reduce the resulting effect of the disorder. Such drugs activate the sympathetic nervous system by way of a baroreceptor reflex to produce an increased heart rate and force of myocardial contraction, which are not beneficial or desirable effects. Other side effects for such drugs include headache, heart palpitations, anxiety, mild depression, myocardial infarction, congestive heart failure, fatigue and weakness. Further, pharmacological effect is not specific in its effect on the initial molecular cause of the disease activity, and treats a limited spectrum of effects in the diseases, which are dependent on several factors.

None of these treatment methods is directed towards the underlying disease processes, the molecular causes of the disease or disorders, or towards restoring the structure and function of the blood vessels to levels that reduce or eliminate the danger posed by cardiovascular diseases. There is no treatment to reduce the level of obstruction in arteries that are not severely occluded or to enhance the arteries normal inherent ability to resist thrombus formation leaving these patients still at significant risk of a heart attack.

In patents, U.S. Pat. Nos. 6,255,296 and ______ to Daniels, the inventor described a novel method and composition that is directed to various cardiovascular diseases. The composition is described as arginine and Heparin or their physiological salts or functional analogs. Though arginine is absorbed by the body in therapeutic amounts clinical effects are observed only at relatively high doses of more than 6 grams per day. Such large doses cause inconvenience to patients due to the number and size of tablets or capsules and eventually causes loss of patient compliance over time. Thereby limiting the potential therapeutic effects of oral arginine and its metabolic byproduct nitric oxide.

In view of the foregoing, there is a significant need for a pharmacological composition and method, and an objective of the present invention, that is directed towards treating the underlying cardiovascular disease process, and towards restoring the structure and improving the functions of the blood vessel cells and in particular the function-structure properties of the endothelium which lines all blood vessels and the heart.

There is also a need for a pharmacological composition, and an objective of the present invention, with enhanced absorption capability, such that the polyarginine in the described composition is absorbed in greater amounts by a patient. There is a further need to increase patient compliance in administration of daily dosages of the composition of the invention.

It is an objective of the present invention to provide a treatment, which is directed to preventing and minimizing dysfunctional atomic and molecular interactions within human cellular matrix or cellular environment, which lead to cardiovascular disease and atherosclerosis.

It is another objective of the present invention to provide a treatment that is directed to retarding adverse consequences of free radicals generated in human cellular matrix. It is also another objective of the present invention to stimulate an increased production of nitric oxide within human cellular matrix or cellular environment.

### SUMMARY

The present invention enhances the absorption of a therapeutically-effective amount of polyarginine by administering a composition comprised of polyarginine, to a patient. The dose of polyarginine in the heparin-polyarginine composition administered to the patient is between approximately 100 mg to 6000 mg daily. Optionally, the dose of polyarginine administered to the patient is between approximately 200 mg to 1900 mg daily. Optionally, the preferred dose of polyarginine administered to the patient is between approximately 400 mg to 1800 mg daily.

The object of the present invention is to improve and enhance effectiveness of the described composition being administered to a patient during a course of treatment.

A method and composition is disclosed for treatment of the mammalian cellular environment in prevention of disease by administering to a patient a therapeutically-effective amount of polyarginine for greater absorption of arginine, and co-administering a therapeutically-effective amount of heparin or its functional analog or their physiologically acceptable salts. The described method is used for the treatment of cardiovascular diseases such as atherosclerosis and arteriosclerosis.

It is understood that optionally heparin and a functional analog are used in conjunction with each other, and/or a functional analog and its physiologically acceptable salts are used together. Optionally, heparin is substituted for by heparan sulfate.

Brown algae may be substituted for heparin as they contain sulfated polysaccharides. The amount of brown algae is sufficient to deliver a therapeutic amount equivalent to 8,000 and 12,000 IU of heparin activity on a daily basis. Nori algae may also be substituted for heparin as it too contains sulfated polysaccharides. The amount of nori algae is sufficient to deliver a therapeutic amount equivalent to 8,000 and 12,000 IU of heparin activity on a daily basis. Nori algae and polyarginine may also be used to form a capsule shell for the delivery of other pharmaceuticals, nutrients, or vitamins and minerals.

An advantage of the method and device of the invention is that the co-administration of a poly-arginine-Heparin Sulfate composition produces beneficial nitric oxide effects (decreased endothelial adhesiveness for inflammatory cells, reduced LDL cholesterol binding, decreased platelet activation, and vasodilation, etc.) at lower administered arginine doses from when mono-arginine is administered. Heparin enhances the activity of endogenous Nitrogen Oxide (eNOS), which leads to increased Nitric Oxide (NO) production at lower arginine doses.

Another advantage of the described composition and method is that polyarginine binds to endogenous and administered heparin and heparans much more avidly than mono-arginine alone. Such binding facilitates heparin absorption and reduces binding of arginine containing peptide domains to endogenous heparans as the sulfate and carboxyl groups on endogenous heparin sulfate are bound to polyarginine and cannot simultaneously bind to other arginine containing substances. This leads to protection of endogenous heparans in their normal physiological roles.

Yet another advantage of a polyarginine composition is that polyarginine produces enhanced trans-membrane absorption of administered heparans. By administering Heparin with polyarginine, more Nitric Oxide is generated by heparan inducing activity of eNOS.

Another advantage is that polyarginine is a substrate for eNOS and produces NO as does mono-arginine.

Finally, the complex formation between polyarginine and heparan sulfate polymers directly applies the polyarginine to the endothelial surfaces rather than distributing the arginine to the plasma.

### BRIEF DESCRIPTION OF DRAWINGS

FIG. 1 is a representation of a long chain heparin polymer 20.

FIG. 2 is a representation of a densified higher polymer.

FIG. 3 is a representation of arginine 22 cross-linking heparin polymers.

FIG. 4 is a representation of a healthy gel matrix comprised of multiple polymers.

FIG. 5 is a representation of holes created by binding of clotting factors and lipoproteins onto binding sites precluding cross-linkage of polymers strands and self-association of strands.

FIG. 6 is a representation of a chemical structure of a heparin polymer.

FIG. 7 is a representation of a chemical structure of a heparin-arginine-heparin cross-linked polymer.

FIG. 8 is a representation of a polyarginine 21 chemical structure.

FIG. 9 is a representation of a bi-lipid layer held between two bodies of heparin-arginine-water gel.

FIG. 10 is a representation of a disrupted bilipid layer, wherein the gel matrix has holes with straying phospholipid molecules therein.

FIG. 11 is a representation of polar plaque forming molecules entering the bilipid layer through openings formed in the heparin-arginine-water gel.

### DETAILED DESCRIPTION

As has been previously described by the inventor, medical literature and thinking is pervasive with the thinking that high cholesterol levels cause occlusion of the coronary and other arteries, which then cause infarction and ischemia. The inventor, in patents, U.S. Pat. Nos. 6,255,296 and ______, outlines the fact that endothelial cell surface thrombosis, rather than cholesterol occlusion is the proximate cause of ischemia and infarction. The inventor's conception is that cholesterol accumulation in arteries results in loss of the surface anti-thrombotic effects of sufficient Nitric Oxide and Heparin Sulfate, which prevent endothelial and artery based surface thrombotic activity.

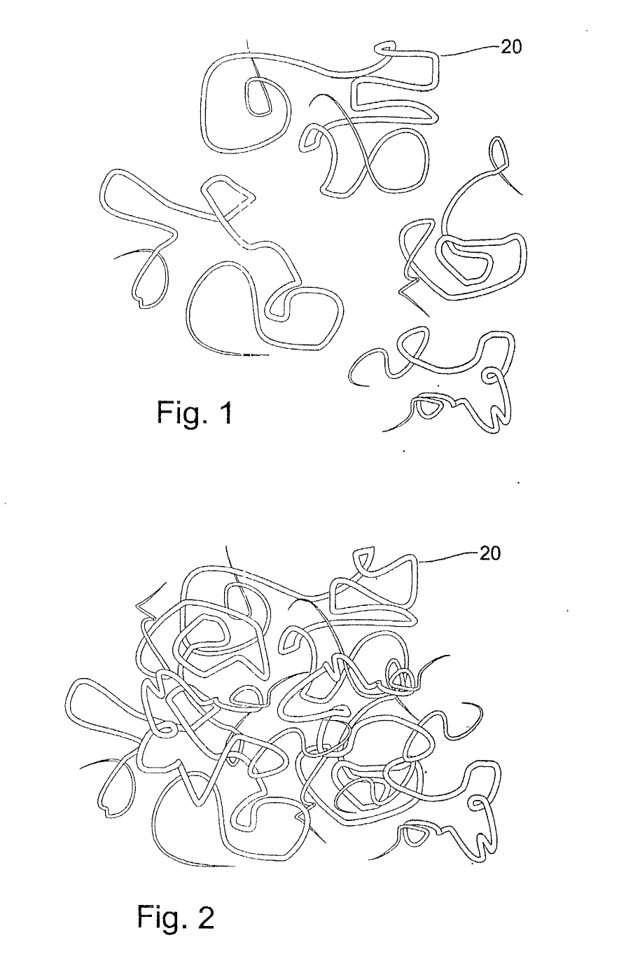

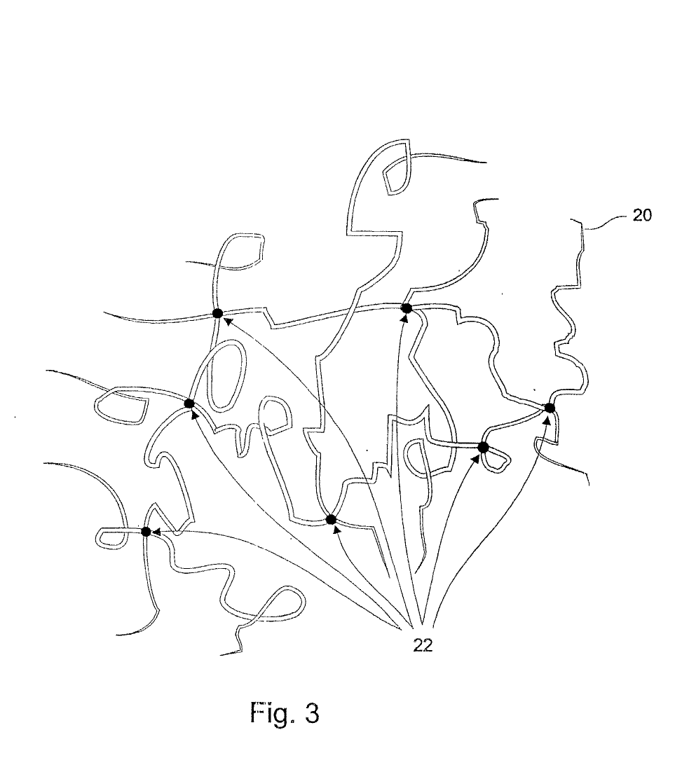

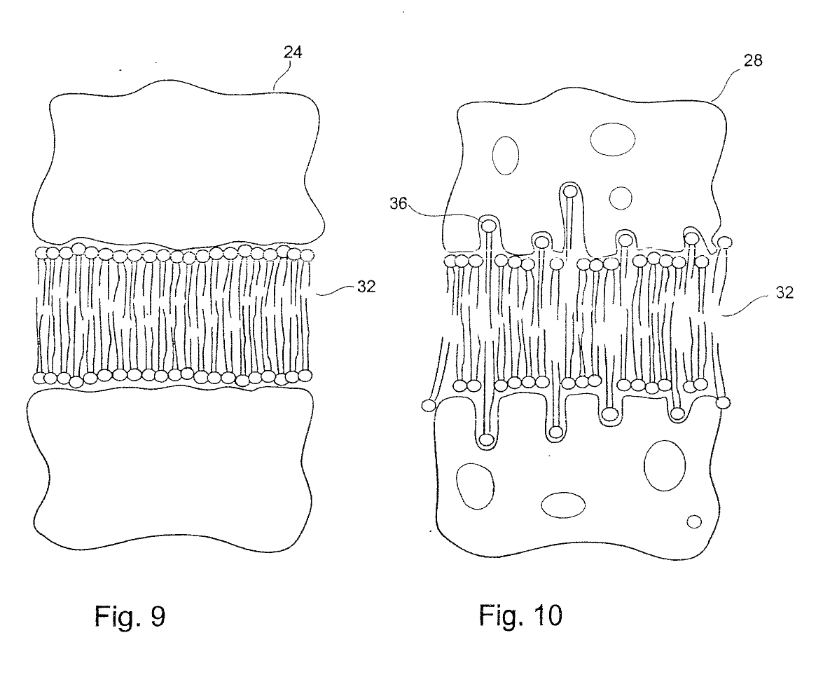

It is also a conception of the inventor that a cellular environment (cellular matrix or gel matrix) composed of charged polymers-highly charged peptide-water polymers, such as heparin-arginine-water, is responsible for controlling the structure and ultimately the function of human cells within this cellular environment. As the human blood vessel is only one cell thick, it too operates within this charged polymers-highly charged peptide-water environment. Thus, this charged polymers-arginine-water environment impacts such important functions of the cells by effecting protein distribution and functionality, cell signaling processes, genetic or DNA-RNA transcription regulation, and the physical/chemical properties of cells, including blood vessel wall cells. FIG. 1 is a representation of long-chain heparin polymer 20 and FIG. 2 represents the polymer in 20 in a densified form. FIG. 3 is a representation of arginine 22 cross-linking heparin polymers 20 to form a matrix. The polymers 20 and 22 organize water into arenas for confining bilipid layer membranes 32, for example, creating cell turgor and form and limiting hydrolytic properties of water on other molecular structures, as shown in FIG. 9.

It should also be noted that heparins or heparin domains within these polymer structures are members of the group commonly referred to as endogenous heparans. Exogenous heparans, including heparin, have functions, which protect the endogenous heparans.

The present invention is directed to a formulation for treatment of the gel matrix and inhibiting cardiovascular disorder or disease and endothelial dysfunction by administering lower dosages of polyarginine as compared to monoarginine. In accordance with the invention, a patient susceptible to or suffering from a cardiovascular disorder or disease such as atherosclerosis, arteriosclerosis, congestive heart failure, angina pectoris, or other diseases associated with the cardiovascular system, is treated with a therapeutically effective amount of a first substance characterized as exogenous heparin or its functional analogs or their physiologically acceptable salts, and a second substance characterized as exogenous polyarginine or its functional analogs or physiologically acceptable salts thereof.

A therapeutically effective amount of heparin activity is defined primarily by clinical response in a patient, and ranges from about 2,000 IU to 200,000 IU daily on variable schedule. A more preferred range of an effective amount of heparin activity is between about 5,000 to 20,000 IU daily on a variable schedule. A most preferred range of an effective amount of heparin activity is between about 8,000 IU and 12,000 IU daily on variable schedule.

For example, the Heparin is characterized such that it should be an amount sufficient to exert cell surface anti-thrombotic effects on the endothelial cells, while not increasing the patient's risk of internal or external hemorrhaging and effectively maintaining integrity and functionality of the cellular membranes and surrounding environments of the endothelial cells.

A therapeutically effective amount of polyarginine 21 ranges from 100 mg to 6,000 mg daily dependent on the underlying condition and nature of physiological processes requiring treatment. A preferred range of administered polyarginine ranges from 200 mg to 1900 mg daily. A more preferred range of polyarginine ranges from 400 mg to 1800 mg daily.

For example, the polyarginine should be a sufficient amount (1) to sustain levels of nitric oxide to keep various cell types from dysfunctional activation states in the patient, (2) to increase prostacyclin secretion, (3) to reduce binding of extra-cellular proteins and heparin binding proteins to endogenous heparans, and (4) to bind to available sulfate and carboxyl groups on heparan in order to decrease random disorganization and reorganization of endogenous heparan polymers.

Again, effective doses of heparin vary with the particular patient condition and the method of administration. For example, it is noticed that subcutaneous injection of heparin results in greater concentration in the cellular and membrane domains than intravenous injection, and it is the inventor's observation that oral heparan sulfates localizes almost exclusively to cell surface membranes, especially the endothelium. Thus, the preferred method of administration of heparin for the present invention is through the oral route, while the least preferred method is via intravenous injection.

Polyarginine, as used herein Poly L-arginine, is preferably co-administered together with or separately from the heparin. Poly L-arginine also includes sulfates thereof and their functional analogs.

The physiological condition of the patient will largely dictate the required dosages and frequencies of polyarginine administration, i.e. weight, age, disease, sex.

The compound of the present invention can be formulated for oral, sublingual, subcutaneous, intravenous, transdermal or rectal administrations in dosages and in admixture with pharmaceutical excipients or vehicles including implantation or controlled-release devices. Furthermore, the compound of the present invention is optionally used, either alone or in conjunction with other material that are currently used as capsules, to form a capsule shell. For example, the compound of heparin and polyarginine can be dispersed in a physiologically acceptable, non-toxic liquid vehicle, such as water. The capsule shell comprised of the compound of the present invention is then used to administer or deliver other pharmaceuticals, nutrients, or vitamins and minerals.

Alternatively, the compound can be given in tablet, capsule, powder, granules or coated tablet form. The compound is made using conventional methods, and may be mixed with conventional pharmaceutical auxiliaries, such as binders, fillers, preservatives, tablet disintegrators, flow regulators, plasticizers, wetting agents, dispersants, emulsifiers, solvents, retarding agents and/or anti-oxidants. It is also optionally contained or complexed with lipids in various formulations and molecular arrangements.

The inventor recognizes as integral to the invention, that cell surface based antithrombotic activity is distinctly different from plasma anti-coagulation. The invention achieves cell based antithrombotic activity without the inhibition of plasma anticoagulant factors. Thus, the invention avoids the risks of spontaneous hemmorhage or excessive bleeding due to vessel injury attendant to plasma anticoagulation with currently available anticoagulant treatments such as Coumadin® and heparin.

Localization of administered heparin or heparin analogues to cell surfaces (e.g. endothelial surfaces) by oral administration inhibits thrombotic activity within and on artery and blood vessel surfaces without the inhibition of plasma clotting factors seen with currently available anticoagulants.

The drawings of the present invention aid to illustrate a polyarginine composition and what are believed to be key polymers and processes which pertain to the aforesaid composition of exogenous heparin and exogenous polyarginine.

An efficiently operating homeostatic system is crucial to cellular function within mammalian organisms. In a healthy state, there is formed a gel matrix of heparans, highly charged peptide, and water polymers, which houses a plurality of other molecules by accommodating dynamic binding of and release of such molecules without reaching concentration levels which destroy the gel structure and its regulatory functionalities.

Commercially, heparin is normally derived from animal tissue such as livers and lungs of cattle, bovine species and sheep. Heparin and heparin-like compounds have also been found in plant tissue where the heparin or heparin-like compound is bound to the plant proteins in the form of a complex. Heparin and heparin-like compound derived from plant tissue are of particular importance because they are considerably less expensive than heparin and heparin-like compounds harvested from animal tissue.

Plants which contain heparin or heparin-like compounds such as physiologically acceptable salts of heparin, or functional analogs thereof will provide a suitable source for the present invention. Typical plant sources of heparin or heparin-like compounds include artemisia princepts, nothogenia fastigia (red seaweed), copallina pililifera (red algas), cladophora sacrilis (green seaweed), chaetomorpha anteninna (green seaweed), aopallina officinalis (red seaweed), monostrom nitidum, laminaria japonica, flipendula ulmaria (meadowsweet), ecklonia kuroma (brown seaweed), ascophyllum nodosum (brown seaweed), ginkgo biloba, ulva rigida (green algae), stichopus japonicus (seacucumber), panax ginseng, spiralina maxima, spirulina platensis, laurencia gemmifera (red seaweed) larix (larchwood), and analogs thereof.

Such plants are considered to be an effective and efficient source of heparin or heparin-like compounds for use in the present invention. It is also understood that whenever this application refers to heparin, it contemplates the use of heparin-like compounds or the functional analogs of heparin instead of heparin.

Polymer strands 20 are an organizing determinant for membranes, proteins, receptors, ion channels, cell organelles, nuclear membranes, membrane pores, and other complex cellular constituents. The polymers 20 and 22 organize water into arenas for confining bilipid layer membranes 32, for example, creating cell turgor and form and limiting hydrolytic properties of water on other molecular structures.

Heparin's 20 high sulfate content imparts a high negative charge which attracts and binds positively charged substances like basic amino acids, basic domains of proteins and peptides, cations, water and other such charged molecules.

Arginine 22 has a high positive charge and strongly associates with heparin along membrane surfaces such as endothelium and basement membranes and in association with water 26, organize as a gel matrix 24.

The gel may be in a constant state of change, including transitions from one state or phase to another. As such, conformation can change and derangements occur as different substances move in and out of the gel and as the gel properties change.

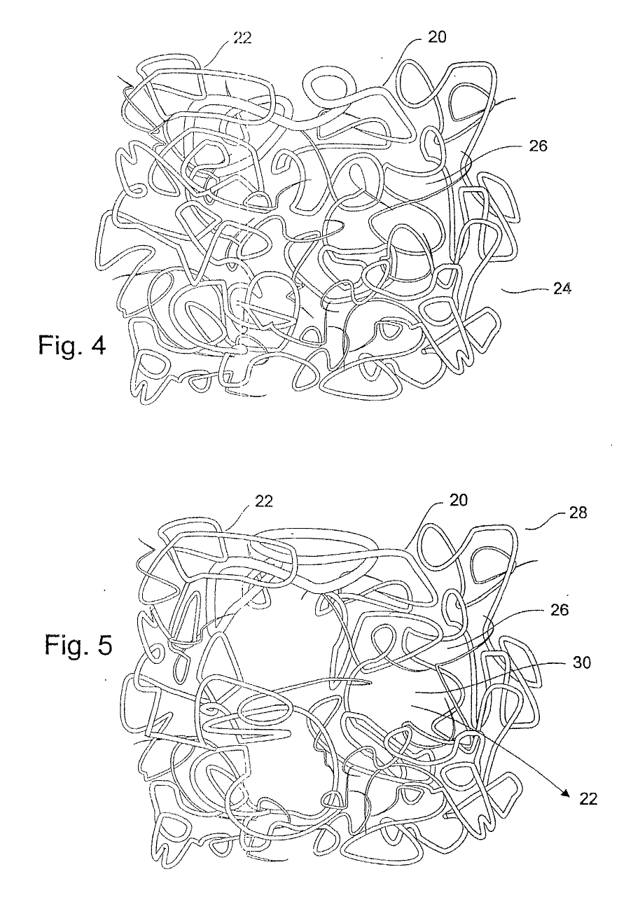

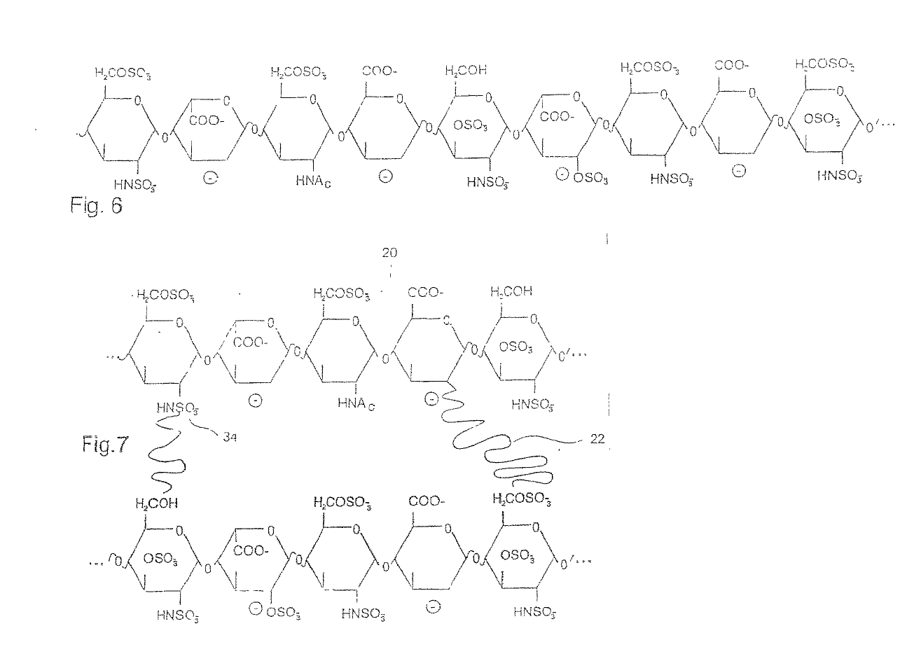

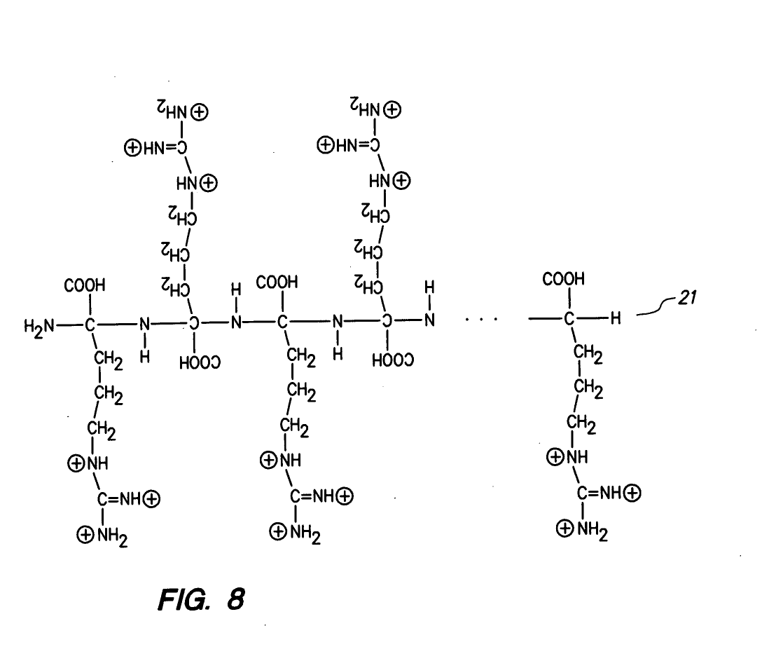

A healthy gel matrix 24, as shown in FIG. 4, is formed from endogenous charged polymers 20, endogenous arginine 22 and water 26. FIG. 5 depicts an unhealthy state of a gel matrix 28 wherein some of the highly charged peptide molecules 22 have been cleaved out of the gel 28. Likewise, charged polymers 20 have been removed from the gel 28. There are thus created gaps between charged polymers 20 into which other molecules can embed or pass through. As seen in FIGS. 6-8, a representative chemical structure of charged polymer 20, charged polymers 20-arginine polymer 22 and highly charged polypeptide (polyarginine) polymer 21 are shown, respectively. Arginine groups 22 are attached to the sulfate sites 34 along the polymer 20, wherein the NHx (e.g. x=1 or 2) groups are positively charged and attach to negatively charged SO3 groups. A higher number of cross-linking bonds which exist between charged polymers 20 equates to a higher densification characteristic of the gel matrix with smaller pores.

The healthy gel structure 24 has a conformation that preferentially supports interaction and binding of foreign molecules. The capacity to accommodate intrusions of such molecules before the gel structure collapses and loses its functionality is an important characteristic of the gel system.

An example of polar molecules that heparin binds and inactivates, thereby modulating their activity, are serine proteases, other clotting factors and thrombolytic agents, antithrombin-thrombin, complement, apo-lipoproteins, growth-promoting factors, mitogens, heparinase, lipoprotein lipase, growth-inhibiting factors, chemotactic factors, super oxide dismutase, cytokines, numerous enzymes, and cytoskeletal proteins such as fibronectin.

As these intrusions accumulate locally or in a distributed fashion, they cause an interference within the gellular association of charged polymers 20 and arginine 22. The interference can cause the gel structure to deteriorate, thus increasing its porosity or collapse altogether in a localized or distributed fashion. In addition, the intrusion may trigger a release of other bound polar molecules, such as calcium which would induce a non-homeostatic event.

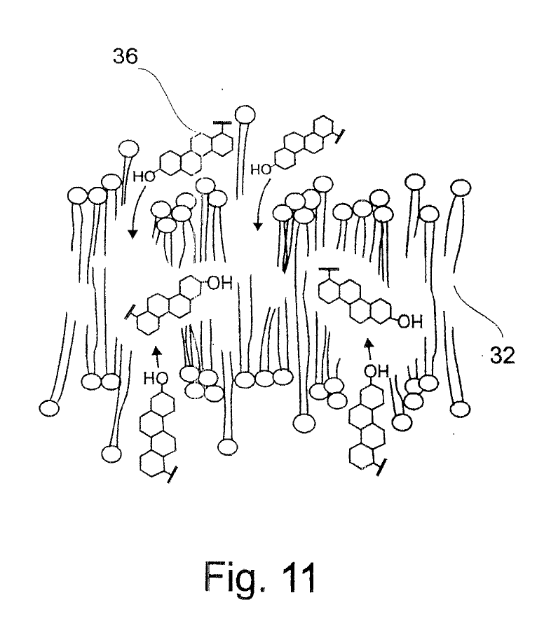

The permeability created by the interference of such molecules allows macromolecules or cells 36 to enter and traverse the gel 28, as shown in FIG. 10. For example, cholesterol, clotting factors and water traverse the gel reaching a bilipid layer, or other subendothelial locations, as seen in FIG. 11. In addition, ionic strength, flow stress, heat, osmotic pressure or other forms of energy transfer to the gel can deteriorate the properties of the gel as described above.

These intrusions result in a displacement of arginine and decreased generation of nitric oxide as an additional effect. Intrusions limit the binding capacity of the charged polymer such as heparin for arginine and other molecules within the gel.

In order to reverse this distruption of the gel matrix caused by the removal of arginine and/or heparin, the present invention employs a composition to maintain and rejuvenate the gel matrix and its functionality. In this regard, the present invention utilizes a full range of molecular weight heparin and lower daily doses of therapeutic amounts of polyarginine to give optimal absorption of arginine as compared to administration of monoarginine, pore closure and stabilization, and number and distribution of binding sites, wherein signaling, anti-proliferation, cell surface anti-thrombotic, and anti-inflammatory effects are maintained. Thus, the homeostasis-promoting functionalities of heparin, arginine, and charged polymers-arginine-water gel matrix, resultant from the herein-described composition, retard continuous and accumulative change and injury to cellular domains. By this retarding effect, cholesterol accumulations, generally referred to as “arterial plaques” are minimized.

Heparin and polyarginine co-administration also leads to increased lipoprotein lipase release and tissue factor pathway inhibitor release, with beneficial effects on plaque stability, growth, rupture, and regression.

The expression of endogenous heparin at the gel surface, generates a signal to the golgi apparatus to produce endogenous heparin. Added exogenous heparin or its functional equivalents accumulates at the blood/endothelium surface thereby reconstituting the prostacyclin receptors, which may have been damaged and depleted over time. Nitric oxide production at or near the same surface occurs from nitric oxide synthase action on exogenous and endogenous arginine substrate. This nitric oxide amplifies the signal by increasing the local concentration of prostacyclin, whose production is mediated by the nitric oxide. Thus, heparin is generated in quantities sufficient to allow reassociation of arginine and heparin and restores the gel structure, as well as releasing or rearranging potentially injurious molecules in the gel matrix.

Addition of exogenous heparin to the gel system protects the functionality of the arginine binding in the gel, and addition of arginine to the gel system protects the functionality of the charged polymers in the gel. In the extragellular medium, the ability of heparin to bind and quiesce molecules is augmented by simultaneous addition of exogenous heparin and exogenous polyarginine, wherein exogenous heparin is binding to extragellular potentially-intruding molecules, thus allowing existing gellular charged polymers to associate with gellular arginine. Exogenous polyarginine becomes the more available substrate for nitric oxide synthase, thereby protecting gellular endogenous arginine from the nitric oxide synthase activity and allowing the endogenous arginine to continuously re-associate with the gellular charged polymers, thus protecting the gel functionality.

Nitric oxide produced from arginine is an important physiological mediator. The enzyme responsible for nitric oxide production, nitric oxide synthase, requires CA++ and Calmodulin. The functionality of the charged polymers-arginine gel includes its binding and regulation of CA++ and Calmodulin. By regulating Calmodulin activity, the charged polymers-arginine gel regulates nitric oxide synthase activity responsible for nitric oxide production.

The binding of water, small anions and cations within the charged polymers-arginine-water gel is facilitated by pi-bonding properties inherent in the saccharide ring structure within the charged polymers. Changes in the shared electron density and electrical charge variation regulated the state of solvation and conformation of the gel polymers. Thus, small anion and cation binding induces changes in the state of solvation, changes in catalytic and hydrolytic properties of water, and changes in capacity of the gel to bind water and other molecules.

Low to high molecular weight heparin, preferably having a high degree of sulfation, can be used as well as standard heparin as is commercially available. Human, animal, and recombinant heparin sources are believed to be useful in practicing the invention and are capable of stimulating the full range of responses claimed herein. The source of exogenous heparin, including the possibility of human recombinant heparin, and the source of polyarginine impart no special or additional properties to the homeostatic functionalities observed for those individual elements or their conjoined, synergistic functionalities. Various glycosaminoglycans, similar to heparin, are subject to in vivo epimerization and sulfation resulting from agents which promote acylation reactions and sulfation reactions, such as acetyl salicylic acid, thereby producing heparin or heparin functionality. Thus, for example, heparin sulfate is considered an analog of heparin. Heparin can be used in the form of its salts with physiologically tolerated bases, for example, sodium, calcium, magnesium, diethylamine, triethylamine or triethanolamine. Promoters of increased heparin production, such as prostacyclin, are the functional equivalent of heparin, as would be analog's thereof, such as taprostene and may be employed in the present invention.

Endothelial cell injury and myocardial cell injury occur from free radicals. Heparin binds super oxide dismutase which absorbs high energy electrons and deactivates free radicals. Heparin and nitric oxide bind free radicals preventing damages to endothelial cells.

Congestive heart failure is in part due to free radical injury to myocardial cells. Heparin, super oxide dismutase and nitric oxide all attack and neutralize free radicals, therefore, diseases associated with cellular injury from free radicals are effectively treated and prevented by the present invention. Also, heparin aids in the reconstruction of damaged tissue by forming a complex with and removing extracellular matrix protein accumulations, e.g. fibronectin with consequent reversal or minimization of organ hypertrophy states. Heparin, via its association with polyarginine, enhances regeneration of endothelium following an injury to an endothelium surface.

It will be readily apparent to those skilled in the art that many modifications, derivations and improvements are within the scope of the invention. Such modifications, derivations, and improvements should be accorded full scope of protection by the claims appended hereto.

1. A method for treatment of the mammalian cellular environment for the prevention of disease comprising the step of administering to a patient a therapeutically-effective amount of polyarginine for greater absorption of arginine, and co-administering a therapeutically-effective amount of heparin or its functional analog or their physiologically acceptable salts.

2. The method of claim 1 wherein the dose of polyarginine administered to the patient is between approximately 100 mg to 6000 mg daily.

3. The method of claim 1 wherein the dose of polyarginine administered to the patient is between approximately 200 mg to 1900 mg daily.

4. The method of claim 1 wherein the dose of polyarginine administered to the patient is between approximately 400 mg to 1800 mg daily.

5. The method of claim 1 wherein the dose of heparin or its functional analog or their physiologically acceptable salts, is between approximately 2,000 IU and 200,000 IU of activity on a daily basis.

6. The method of claim 1 wherein the dose of heparin or its functional analog or their physiologically acceptable salts, is between approximately 5,000 IU and 20,000 IU of activity on a daily basis.

7. The method of claim 1 wherein the dose of heparin or its functional analog or their physiologically acceptable salts, is between approximately 8,000 IU and 12,000 IU of activity on a daily basis.

8. The method of claim 1 wherein heparin and a functional analog is used.

9. The method of claim 1 wherein a functional analog and its physiologically acceptable salts is used.

10. The method of claim 1 wherein heparin is substituted for by heparan sulfate.

11. The method of claim 1 wherein heparin is substituted for by brown algae.

12. The method of claim 11, wherein the amount of brown algae is sufficient to deliver a therapeutic amount equivalent to 8,000 and 12,000 IU of heparin activity on a daily basis.

13. The method of claim 1 wherein heparin is substituted for by nori algae.

14. The method of claim 13, wherein the amount of nori algae is sufficient to deliver a therapeutic amount equivalent to 8,000 and 12,000 IU of heparin activity on a daily basis.

15. The method of claim 13 wherein the nori algae and polyarginine are used to form a capsule shell for the delivery of other pharmaceuticals, nutrients, or vitamins and minerals.

16. The method of claim 1 wherein the disease is cardiovascular disease.

17. The method of claim 1 wherein the disease is atheresclerosis.

18. The method of claim 1 wherein the disease is arterioscelrosis.

19. A composition with enhanced absorption of arginine in humans for treatment of cardiovascular disease comprising a mixture of heparin or its functional analog or their physiologically acceptable salts, and polyarginine, in therapeutic proportions.

20. The composition of claim 19 comprising a mixture of functional analogs or physiologically acceptable salts of polyarginine.

21. The composition of claim 19 wherein the dose of polyarginine administered to the patient is between approximately 100 mg to 6000 mg daily.

22. The composition of claim 19 wherein the dose of polyarginine administered to the patient is between approximately 200 mg to 1900 mg daily.

23. The composition of claim 19 wherein the dose of polyarginine administered to the patient is between approximately 400 mg to 1800 mg daily.

24. The composition of claim 19 wherein the dose of heparin activity is between approximately 2,000 IU and 200,000 IU on a daily basis.

25. The composition of claim 19 wherein the dose of heparin activity is between approximately 5,000 IU and 20,000 IU on a daily basis.

26. The composition of claim 19 wherein the dose of heparin activity is between approximately 8,000 IU and 12,000 IU on a daily basis.

27. The method of claim 19 wherein heparin and a functional analog is used.

28. The method of claim 19 wherein a functional analog and its physiologically acceptable salt is used.

29. The composition of claim 19 wherein heparin is substituted for by heparan sulfate.

30. The composition of claim 19 wherein heparin is substituted for by brown algae.

31. The composition of claim 30, wherein the amount of brown algae is sufficient to deliver a therapeutic amount equivalent to 8,000 and 12,000 IU of heparin activity on a daily basis.

32. The composition of claim 19 wherein heparin is substituted for by nori algae.

33. The composition of claim 32, wherein the amount of nori algae is sufficient to deliver a therapeutic amount equivalent to 8,000 and 12,000 IU of heparin activity on a daily basis.

34. The composition of claim 32 wherein the nori algae and polyarginine are used to form a capsule shell for the delivery of other pharmaceuticals, nutrients, or vitamins and minerals.

35. A composition with enhanced absorption of arginine in treatment of the mammalian cellular environment for the prevention of disease comprising a mixture of heparin or its functional analog, or their physiologically acceptable salts, and polyarginine, in therapeutic proportions.

36. The composition of claim 35 comprising a mixture of functional analogs or physiological acceptable salts of polyarginine.

37. The composition of claim 35 wherein heparin is substituted for by heparan sulfate.

38. The composition of claim 35 wherein brown algae is substituted for the heparin.

39. The composition of claim 38 wherein the brown algae and polyarginine are used to form a capsule shell for the delivery of other pharmaceuticals, nutrients, or vitamins and minerals.

40. The composition of claim 38, wherein the amount of brown algae is sufficient to deliver a therapeutic amount equivalent to 8,000 and 12,000 IU of heparin activity on a daily basis.

41. The composition of claim 35 wherein nori algae is substituted for the heparin.

42. The composition of claim 41 wherein the nori algae and polyarginine are used to form a capsule shell for the delivery of other pharmaceuticals, nutrients, or vitamins and minerals.

43. The composition of claim 41, wherein the amount of nori algae is sufficient to deliver a therapeutic amount equivalent to 8,000 and 12,000 IU of heparin activity on a daily basis.

44. The method of claim 35 wherein the disease is cardiovascular disease.

45. The method of claim 35 wherein the disease is atherescierosis.

46. The method of claim 35 wherein the disease is arterioscelrosis.
